(19) United States
(12) Patent Application Publication
(10) Pub. No.: US 2025/0072735 A1
(43) Pub. Date: Mar. 6, 2025

(54) FLUID DELIVERY DEVICE FOR USE WITH AN ENDOSCOPE

(71) Applicant: Boston Scientific Medical Device Limited, Galway (IE)

(72) Inventors: Deepak Kumar Sharma, Muzaffarnagar (IN); Sharath Kumar G, Ramanagar District (IN); Nachiket Gole, Pune (IN)

(73) Assignee: Boston Scientific Medical Device Limited, Galway (IE)

(21) Appl. No.: 18/825,318

(22) Filed: Sep. 5, 2024

Related U.S. Application Data
(60) Provisional application No. 63/580,839, filed on Sep. 6, 2023.

(51) Int. Cl. A61B 1/015; A61B 1/00; A61B 17/00

(52) U.S. Cl. CPC A61B 1/015; A61B 1/00064; A61B 1/00133; A61B 1/00135; A61B 17/00491

(57) ABSTRACT

Systems for use with an endoscope are disclosed. An example system may include a sleeve configured to be coupled to an outer surface of an endoscope. A housing may be coupled to the sleeve. The housing may have a proximal end region, a distal end region, and a fluid chamber defined therein. A plurality of openings may be formed in the distal end region. An actuator may be coupled to the proximal end region of the housing and extend proximally therefrom. A distal ring member may be disposed within the housing. The distal ring member may be axially fixed relative to the sleeve and relative to the housing. The housing is configured to shift relative to the endoscope between a first position and a second position.

### CROSS-REFERENCE TO RELATED APPLICATIONS

This application claims the benefit of U.S. Provisional Patent Application Ser. No. 63/580,839 filed on Sep. 6, 2023, the disclosure of which is incorporated herein by reference.

### TECHNICAL FIELD

The present disclosure pertains to medical devices, and methods for manufacturing medical devices. More particularly, the present disclosure pertains to fluid delivery devices for use with an endoscope.

### BACKGROUND

A wide variety of medical devices have been developed for medical use, for example, intravascular use. Some of these devices include guidewires, catheters, endoscopes, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A system for use with an endoscope is disclosed. The system comprises: a sleeve configured to be coupled to an outer surface of an endoscope; a housing coupled to the sleeve, the housing having a proximal end region, a distal end region, and a fluid chamber defined therein; wherein a plurality of openings are formed in the distal end region; an actuator coupled to the proximal end region of the housing and extending proximally therefrom; a distal ring member disposed within the housing, the distal ring member being axially fixed relative to the sleeve and relative to the housing; and wherein the housing is configured to shift relative to the endoscope between a first position and a second position.

Alternatively or additionally to any of the embodiments above, the sleeve comprises silicone.

Alternatively or additionally to any of the embodiments above, the housing includes an inner wall and an outer wall, and wherein the fluid chamber is defined between the inner wall and the outer wall.

Alternatively or additionally to any of the embodiments above, the distal end region of the housing has an arcuate shape.

Alternatively or additionally to any of the embodiments above, the actuator includes a pull wire.

Alternatively or additionally to any of the embodiments above, further comprising one or more additional actuators.

Alternatively or additionally to any of the embodiments above, further comprising a proximal ring member configured to be disposed about the endoscope and positioned proximal of the distal ring member.

Alternatively or additionally to any of the embodiments above, further comprising a spacer member extending between the distal ring member and the proximal ring member.

Alternatively or additionally to any of the embodiments above, wherein the actuator extends through the proximal ring member.

Alternatively or additionally to any of the embodiments above, further comprising one or more coil members configured to be disposed about the endoscope.

Alternatively or additionally to any of the embodiments above, the actuator is disposed between the one or more coil members and the outer surface of the endoscope.

Alternatively or additionally to any of the embodiments above, the distal ring member is disposed adjacent to the proximal end region of the housing when the housing is in the first position.

Alternatively or additionally to any of the embodiments above, the distal ring member is disposed distally of the proximal end region of the housing when the housing is in the second position.

A system for use with an endoscope is disclosed. The system comprises: a sleeve configured to be coupled to an outer surface of an endoscope; a fluid casing coupled to the sleeve, the fluid casing including an inner wall member, an outer wall member, a fluid chamber defined between the inner wall member and the outer wall member, and an arcuate distal face having a plurality of openings formed therein; a distal ring member disposed within the fluid casing, the distal ring member being axially fixed relative to the endoscope; wherein the fluid casing is configured to shift between a first position and a second position; wherein the distal ring member is disposed adjacent to a proximal end region of the fluid casing when the fluid casing is in the first position; wherein the distal ring member is disposed distally of the proximal end region of the fluid casing when the fluid casing is in the second position; and an actuator coupled to the fluid casing and extending proximally therefrom, the actuator being configured to shift the fluid casing between the first position and the second position.

Alternatively or additionally to any of the embodiments above, further comprising a proximal ring member configured to be disposed about the endoscope and positioned proximal of the distal ring member.

Alternatively or additionally to any of the embodiments above, further comprising a spacer member extending between the distal ring member and the proximal ring member.

Alternatively or additionally to any of the embodiments above, the actuator extends through the proximal ring member.

Alternatively or additionally to any of the embodiments above, further comprising one or more coil members configured to be disposed about the endoscope.

Alternatively or additionally to any of the embodiments above, the actuator is disposed between the one or more coil members and the outer surface of the endoscope.

A method for delivering fluid during an endoscopic procedure is disclosed. The method comprises: disposing an endoscope having a fluid delivery system coupled thereto adjacent to a target site; wherein the fluid delivery system comprises: a sleeve configured to be coupled to an outer surface of the endoscope, a fluid casing coupled to the sleeve, the fluid casing including an inner wall member, an outer wall member, a fluid chamber defined between the inner wall member and the outer wall member, and an arcuate distal face having a plurality of openings formed therein, a distal ring member disposed within the fluid casing, the distal ring member being axially fixed relative to the endoscope, wherein the fluid casing is configured to shift between a first position and a second position, wherein the distal ring member is disposed adjacent to a proximal end region of the fluid casing when the fluid casing is in the first position, wherein the distal ring member is disposed distally of the proximal end region of the fluid casing when the fluid casing is in the second position, and an actuator coupled to the fluid casing and extending proximally therefrom, the actuator being configured to shift the fluid casing between the first position and the second position; and actuating the actuator to shift the fluid casing toward the second position and to deliver a fluid disposed in the fluid chamber through the plurality of openings in the arcuate distal face of the fluid casing an out from the fluid casing.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of an example fluid delivery device for use with an endoscope.

FIG. 2 is a partial cross-sectional side view of a portion of an example fluid delivery device for use with an endoscope.

FIG. 3 is a partial cross-sectional side view of a portion of an example fluid delivery device for use with an endoscope.

FIG. 4 is a perspective view of a portion of an example fluid delivery device for use with an endoscope.

FIG. 5 is a perspective view of an example fluid delivery device for use with an endoscope.

FIG. 6 is a partial cross-sectional side view of a portion of an example fluid delivery device for use with an endoscope.

FIG. 7 is a partial cross-sectional side view of a portion of an example fluid delivery device for use with an endoscope.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Endoscopes are widely used to treat and/or diagnose a number of different conditions. Some of these conditions (e.g., such as bleeding ulcers) may be treated by using a syringe along with an endoscope to delivery a fluid to the treatment site. It can be appreciated that delivery of fluid in conjunction with an endoscope may be challenging. Disclosed herein are systems and devices for delivery of fluid with (e.g., in conjunction with) an endoscope.

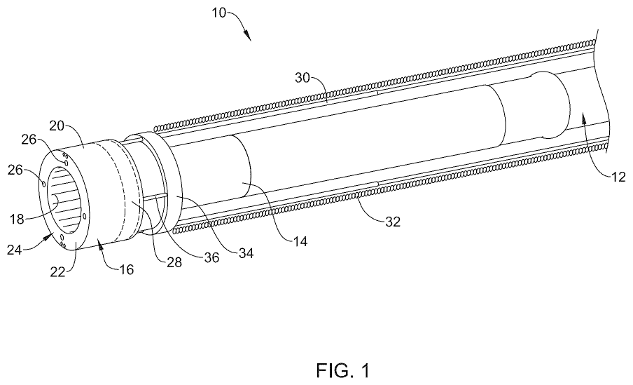

FIG. 1 illustrates an example system 10 for use with an endoscope 12. The system 10 may be secured to the endoscope 12 and can be used, for example, to deliver a fluid to a target. In at least some instances, the target may be bleeding ulcers. Thus, the system 10 may deliver a fluid and/or adhesive patch (e.g., a fluid or fluid-like substance that forms an adhesive patch) that can, for example, help to cover bleeding ulcers, slow or stop bleeding, reduce perforations, reduce stricture formation, combinations thereof, and/or the like. In addition to treating bleeding ulcers, the system 10 may be used to treat patient following a tissue resection procedure (e.g., endoscopic mucosal resection, endoscopic submucosal dissection, polypectomy, etc.), to reinforce a suture line, to seal a fistula, to treat or close a peroral endoscopic myotomy site, and/or the like. A number of additional interventions are contemplated for the use of the system 10.

The system 10 may include a sleeve 14. In general, the sleeve 14 may be configured to be coupled to and/or disposed along an outer surface of the endoscope 12. In some instances, the sleeve 14 may comprise silicone. Other materials are contemplated. The sleeve 14 may help to secure (e.g., frictionally secure) the system 10 to the endoscope 12. Other mechanisms for securing the system 10 to the endoscope 12 are contemplated including mechanical bonds, adhesive bonds, thermal bonds, combinations thereof, and/or the like. In some instances, the system 10 may be permanently affixed to the endoscope 12. In such instances, the system 10 may be inclusive of the endoscope 12. In other instances, the system 10 may be attachable to an endoscope 12. In other words, the system 10 may be an additional component that can be added to an endoscope 12.

A fluid casing or housing 16 may be coupled to the sleeve 14. The fluid casing 16 may include an inner wall 18 and an outer wall 20. A fluid chamber 22 may be defined within the fluid casing 16, for example between the inner wall 18 and the outer wall 20. The fluid casing 16 may have a distal end region and/or distal face 24. In some instances, the distal face 24 may be arcuate. One or more openings 26 may be formed in the distal face 24.

In general, the fluid casing 16 may be configured to house a fluid for delivery to a suitable target location. For example, for interventions aimed at treating bleeding ulcers, one or more therapeutic fluids may be housed in the fluid chamber 22. An example fluid may include an acrylate, a methacrylate, n-butyl 2-cyanoacrylate, other materials and/or adhesives, and/or the lie. Such fluid may be present as a monomer within the fluid chamber 22 and, upon delivery and/or contact with tissue, the fluid may polymerize to bond wound tissue. Other fluids are contemplated. In at least some instances, the fluid may be biocompatible, at least somewhat visible and can be visualized during an intervention, efficacious and/or effective for treating a suitable condition (e.g., cover bleeding ulcer, stop bleeding, etc.), be relatively flowable and/or easy to apply, relatively easy to delivery along the digestive tract, combinations thereof, and/or the like. In some instances, the fluid may form and/or otherwise take the form of an adhesive patch.

In some instances, the fluid may include multiple parts or components. In such instances, the two parts may be kept separate by a partition (not shown) within the fluid chamber 22. Delivery of the two parts may cause the parts to mix together and/or combine to form a therapeutic material. For example, one of the components may include a fibrinogen (e.g., lyophilized pooled human fibrinogen) and the other may include a thrombin (e.g., bovine, human, etc.). In some instances, one or both of the components may include calcium salts. A number of different parts/components are contemplated.

The system 10 may include a distal ring member 28. In at least some instances, the distal ring member 28 may be disposed within the fluid casing 16. The distal ring member 28 may be axially fixed relative to the sleeve 14 and/or relative to the fluid casing 16. As will be described in more detail herein, the distal ring member 28 may function similarly to a plunger or an end of a plunger of a syringe. For example, because the distal ring member 28 may be axially fixed relative to the fluid casing 16, movement or translation of the fluid casing 16 may shift the distal ring member 28 within the fluid casing 16 in a manner that can help to push a fluid contained in the fluid chamber 22 out of the fluid casing 16 via the openings 26.

An actuator 30 may be coupled to the fluid casing 16. For example, the actuator 30 may be coupled to a proximal end region of the fluid casing 16 and extend proximally therefrom. In some instances, the actuator 30 may be a pull wire. The pull wire 30 may be configured to shift the fluid casing 16. In some instances, the system 10 may include one actuator 30. In other instances, the system 10 may include one or more additional actuators 30.

One or more coil members 32 may be disposed along the outer surface of the endoscope 12. For example, the one or more coil members 32 may wrap around the endoscope 12 and the actuator(s) 30. Accordingly, the actuator 30 may extend between the one or more coil members 32 and the outer surface of the endoscope 12. Alternatively, the one or more coil members 32 may wrap around each of the actuators 30 and extend proximally along the actuators 30.

In some instances, the system 10 may include a proximal ring member 34. The proximal ring member 34 may be disposed proximal of the distal ring member 28. In some instances, the actuator 30 may extend through the proximal ring member 34. A spacer member 36 may extend between the distal ring member 28 and the proximal ring member 34. The proximal ring member 34, the distal ring member 28, and the spacer member 36 may be axially fixed relative to one another. For example, the proximal ring member 34 may be axially fixed relative to the distal ring member 28 by the spacer member 36.

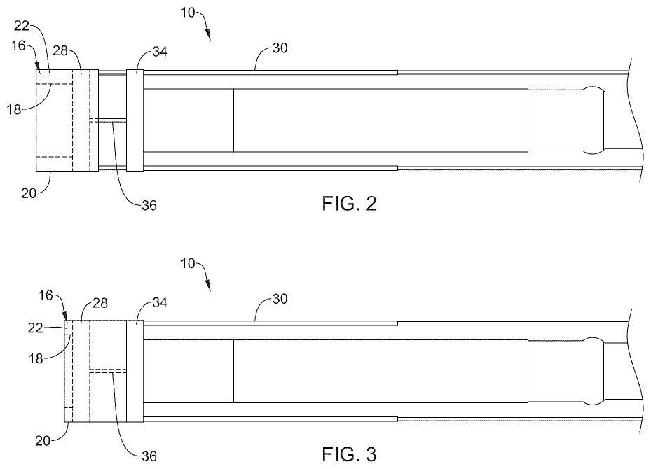

As indicated herein, the fluid casing 16 may be configured to shift relative to the endoscope 12 between a first position and a second position as depicted in FIGS. 2-3. For example, the distal ring member 28 may be disposed adjacent to the proximal end region of the fluid casing 16 when the fluid casing 16 is in the first position as shown in FIG. 2. The distal ring member 28 may be disposed distally of the proximal end region of the fluid casing 16 when the fluid casing 16 is in the second position as shown in FIG. 3. Because the fluid casing 16 is movable relative to the distal ring member 28, the distal ring member 28 may shift within the fluid casing 16 and act like a plunger to inject fluid from the fluid casing 16 through the openings 26.

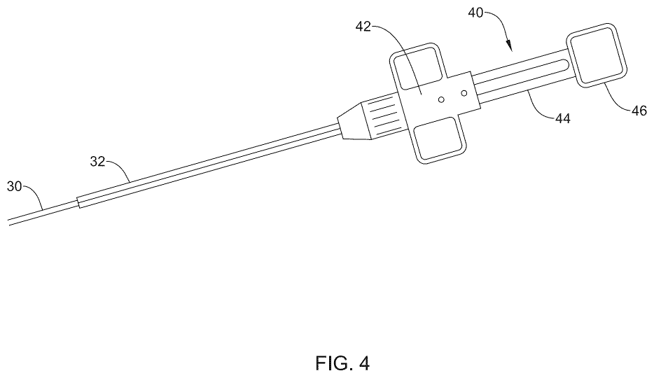

FIG. 4 illustrates a handle 40 coupled to the actuator 30. In general, the handle 40 can be used to shift the actuator 30 and, thus, shift the fluid casing 16. The handle 40 may include a finger pull member 42 slidable along a stem 44. A thumb member 46 may be disposed at the proximal end of the handle 40. The actuator 30 may be coupled to the finger pull member 42 so that retracting the finger pull member 42 along the stem 44 retracts the actuator 30 and, thus, the fluid casing 16. As indicated above, the one or more coil members 32 may be disposed around the endoscope 12 or, as depicted in FIG. 4, the one or more coil members 32 may be wrapped around the actuator 30. In FIG. 4, the one or more coil members 32 are shown with the actuator 30 extending distally therefrom. In practice, the one or more coil members 32 may extend from the handle 40 to the proximal ring member 34.

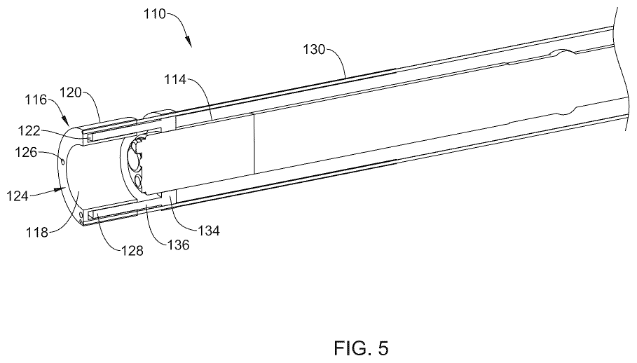

FIG. 5 illustrates another example system 110, similar in form and function to other systems disclosed herein, for use with an endoscope 12. The system 110 may include a sleeve 114. In general, the sleeve 114 may be configured to be coupled to and/or disposed along an outer surface of the endoscope 12. In some instances, the sleeve 114 may comprise silicone. Other materials are contemplated. The sleeve 114 may help to secure (e.g., frictionally secure) the system 110 to the endoscope 12. Other mechanisms for securing the system 110 to the endoscope 12 are contemplated including mechanical bonds, adhesive bonds, thermal bonds, combinations thereof, and/or the like. In some instances, the system 110 may be permanently affixed to the endoscope 12. In such instances, the system 110 may be inclusive of the endoscope 12. In other instances, the system 110 may be attachable to an endoscope 12. In other words, the system 110 may be an additional component that can be added to an endoscope 12.

A fluid casing or housing 116 may be coupled to the sleeve 114. The fluid casing 116 may include an inner wall 118 and an outer wall 120. A fluid chamber 122 may be defined within the fluid casing 116, for example between the inner wall 118 and the outer wall 120. The fluid casing 116 may have a distal end region and/or distal face 124.

In some instances, the distal face 124 may be arcuate. One or more openings 126 may be formed in the distal face 124. Like the fluid casing 16, the fluid casing 116 may be configured to house a fluid for delivery to a suitable target location.

The system 110 may include a distal ring member or region 128. In at least some instances, the distal ring region 128 may be disposed within the fluid casing 116. The distal ring region 128 may be axially fixed relative to the sleeve 114 and/or relative to the fluid casing 116. As will be described in more detail herein, the distal ring region 128 may function similarly to a plunger or an end of a plunger. For example, because the distal ring region 128 may be axially fixed relative to the fluid casing 116, movement or translation of the fluid casing 116 may shift the distal ring region 128 within the fluid casing 116 in a manner that can help to push a fluid contained in the fluid chamber 122 out of the fluid casing 116 via the openings 26.

An actuator 130 may be coupled to the fluid casing 116. For example, the actuator 130 may be coupled to a proximal end region of the fluid casing 116 and extend proximally therefrom. In some instances, the actuator 130 may be a pull wire. The pull wire 130 may be configured to shift the fluid casing 116. In some instances, the system 110 may include one actuator 130. In other instances, the system 110 may include one or more additional actuators 130. In some instances, one or more coil members (not shown) may be disposed along the outer surface of the endoscope 12 and/or wrap around each of the actuators 130.

In some instances, the system 110 may include a proximal ring member or region 134. The proximal ring region 134 may be disposed proximal of the distal ring region 128. In some instances, the actuator 130 may extend through the proximal ring region 134. A spacer member or region 136 may extend between the distal ring region 128 and the proximal ring region 134. The proximal ring region 134, the distal ring region 128, and the spacer region 136 may be axially fixed relative to one another.

In some instances, the proximal ring region 134, the spacer region 136, and the distal ring region 128 may be a singular structure having three sections. For example, the proximal ring region 134 may have a widened outer diameter relative to the remaining section and may be disposed proximally of the fluid casing 116. The distal ring region 128 may be disposed within the fluid casing 116. The spacer region 136 may extend between and couple the proximal ring region 134 with the distal ring region 128.

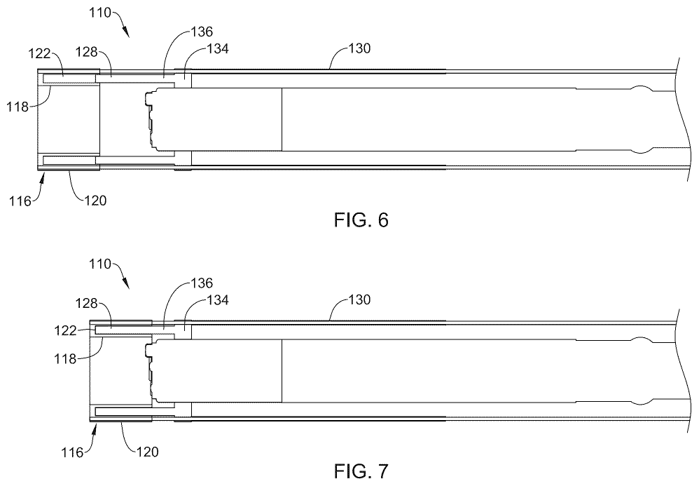

As indicated herein, the fluid casing 116 may be configured to shift relative to the endoscope 12 between a first position and a second position as depicted in FIGS. 6-7. For example, the distal ring region 128 may be disposed adjacent to the proximal end region of the fluid casing 116 when the fluid casing 116 is in the first position as shown in FIG. 6. The distal ring region 128 may be disposed distally of the proximal end region of the fluid casing 116 when the fluid casing 116 is in the second position as shown in FIG. 7. Because the fluid casing 116 is movable relative to the distal ring region 128, the distal ring region 128 may shift within the fluid casing 116 and act like a plunger to inject fluid from the fluid casing 116 through the openings 126.

The materials that can be used for the various components of the system 10 (and/or other systems disclosed herein) may include those commonly associated with medical devices. For example, the system may include components made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for use with an endoscope, the system comprising:
a sleeve configured to be coupled to an outer surface of an endoscope;
a housing coupled to the sleeve, the housing having a proximal end region, a distal end region, and a fluid chamber defined therein;
wherein a plurality of openings are formed in the distal end region;
an actuator coupled to the proximal end region of the housing and extending proximally therefrom;
a distal ring member disposed within the housing, the distal ring member being axially fixed relative to the sleeve and relative to the housing; and
wherein the housing is configured to shift relative to the endoscope between a first position and a second position.

2. The system of claim 1, wherein the sleeve comprises silicone.

3. The system of claim 1, wherein the housing includes an inner wall and an outer wall, and wherein the fluid chamber is defined between the inner wall and the outer wall.

4. The system of claim 1, wherein the distal end region of the housing has an arcuate shape.

5. The system of claim 1, wherein the actuator includes a pull wire.

6. The system of claim 1, further comprising one or more additional actuators.

7. The system of claim 1, further comprising a proximal ring member configured to be disposed about the endoscope and positioned proximal of the distal ring member.

8. The system of claim 7, further comprising a spacer member extending between the distal ring member and the proximal ring member.

9. The system of claim 7, wherein the actuator extends through the proximal ring member.

10. The system of claim 1, further comprising one or more coil members configured to be disposed about the endoscope.

11. The system of claim 10, wherein the actuator is disposed between the one or more coil members and the outer surface of the endoscope.

12. The system of claim 1, wherein the distal ring member is disposed adjacent to the proximal end region of the housing when the housing is in the first position.

13. The system of claim 12, wherein the distal ring member is disposed distally of the proximal end region of the housing when the housing is in the second position.

14. A system for use with an endoscope, the system comprising:
a sleeve configured to be coupled to an outer surface of an endoscope;
a fluid casing coupled to the sleeve, the fluid casing including an inner wall member, an outer wall member, a fluid chamber defined between the inner wall member and the outer wall member, and an arcuate distal face having a plurality of openings formed therein;
a distal ring member disposed within the fluid casing, the distal ring member being axially fixed relative to the endoscope;
wherein the fluid casing is configured to shift between a first position and a second position;
wherein the distal ring member is disposed adjacent to a proximal end region of the fluid casing when the fluid casing is in the first position;
wherein the distal ring member is disposed distally of the proximal end region of the fluid casing when the fluid casing is in the second position; and
an actuator coupled to the fluid casing and extending proximally therefrom, the actuator being configured to shift the fluid casing between the first position and the second position.

15. The system of claim 14, further comprising a proximal ring member configured to be disposed about the endoscope and positioned proximal of the distal ring member.

16. The system of claim 15, further comprising a spacer member extending between the distal ring member and the proximal ring member.

17. The system of claim 15, wherein the actuator extends through the proximal ring member.

18. The system of claim 15, further comprising one or more coil members configured to be disposed about the endoscope.

19. The system of claim 18, wherein the actuator is disposed between the one or more coil members and the outer surface of the endoscope.

20. A method for delivering fluid during an endoscopic procedure, the method comprising:
disposing an endoscope having a fluid delivery system coupled thereto adjacent to a target site;
wherein the fluid delivery system comprises:
a sleeve configured to be coupled to an outer surface of the endoscope,
a fluid casing coupled to the sleeve, the fluid casing including an inner wall member, an outer wall member, a fluid chamber defined between the inner wall member and the outer wall member, and an arcuate distal face having a plurality of openings formed therein,
a distal ring member disposed within the fluid casing, the distal ring member being axially fixed relative to the endoscope,
wherein the fluid casing is configured to shift between a first position and a second position,
wherein the distal ring member is disposed adjacent to a proximal end region of the fluid casing when the fluid casing is in the first position,
wherein the distal ring member is disposed distally of the proximal end region of the fluid casing when the fluid casing is in the second position, and
an actuator coupled to the fluid casing and extending proximally therefrom, the actuator being configured to shift the fluid casing between the first position and the second position; and
actuating the actuator to shift the fluid casing toward the second position and to deliver a fluid disposed in the fluid chamber through the plurality of openings in the arcuate distal face of the fluid casing an out from the fluid casing.
